(19) United States
(12) Patent Application Publication
(10) Pub. No.: US 2026/0309601 A1
(43) Pub. Date: Oct. 8, 2026

(54) PRECIPITATION SOIL EROSION AND MICROBIAL RESPIRATION MONITORING DEVICE

(71) Applicant: Zhengzhou University, Zhengzhou (CN)

(72) Inventors: Zhilei YU, Zhengzhou (CN); Dengming YAN, Zhengzhou (CN); Yuheng YANG, Zhengzhou (CN); Zhe YUAN, Zhengzhou (CN); Huiliang WANG, Zhengzhou (CN); Kai LI, Zhengzhou (CN); Tianye WANG, Zhengzhou (CN); Baisha WENG, Zhengzhou (CN); Tianling QIN, Zhengzhou (CN); Denghua YAN, Zhengzhou (CN)

(21) Appl. No.: 19/679,630

(22) Filed: May 16, 2026

(30) Foreign Application Priority Data
Mar. 25, 2026 (CN) 202610377774.9

(51) Int. Cl. G01N 3/56; G01N 1/08; G01N 17/00; G01N 33/00; G01N 33/24; G01W 1/14

(52) U.S. Cl. CPC G01N 3/567; G01N 1/08; G01N 17/002; G01N 33/004; G01N 33/246; G01W 1/14; G01N 2203/0242; G01N 2203/0246

(57) ABSTRACT

The disclosure discloses a precipitation soil erosion and microbial respiration monitoring device, which belongs to the technical field of monitoring devices, comprising a precipitation regulation unit, an erosion collection unit, a precipitation monitoring unit, and a microbial respiration monitoring unit. The precipitation monitoring unit is arranged on the side of the precipitation regulation unit, the erosion collection unit is arranged below the side of the precipitation regulation unit, the precipitation regulation unit is configured to adjust the precipitation to simulate the erosion of different precipitation on the soil, and the microbial respiration monitoring unit is configured to monitor the microbial respiration in the soil. The disclosure uses the above device to realize the simultaneous monitoring of soil erosion and microbial respiration, and provides basic data for an in-depth understanding of soil environmental dynamics.

### TECHNICAL FIELD

The disclosure relates to the technical field of monitoring devices, in particular to a precipitation soil erosion and microbial respiration monitoring device.

### BACKGROUND

Climate change has a profound impact on the soil environment, manifesting primarily in two aspects: soil erosion and soil microbial activity. Precipitation, as one of the main drivers of soil erosion, plays a particularly significant role in these regions. Changes in precipitation directly influence the extent of soil erosion, which in turn affects soil quality and the health of ecosystems. Meanwhile, soil microbial respiration, a key process in the cycling of soil organic matter and the transformation of nutrients, is essential for understanding and predicting how soils respond to climate change.

Under varying climatic conditions and complex geographical settings, the effective monitoring of soil erosion and microbial activity presents considerable challenges. Traditional soil monitoring methods often require substantial human and material resources, making them difficult to implement in such areas. Moreover, existing monitoring equipment typically fails to account for both soil erosion and microbial respiration simultaneously, creating a gap in the comprehensive assessment of soil environments.

Therefore, there is an urgent need to develop a device capable of monitoring both soil erosion and microbial respiration at the same time.

### SUMMARY

The purpose of the disclosure is to provide a precipitation soil erosion and microbial respiration monitoring device to solve the problems mentioned in the background technology.

In order to achieve the above purpose, the disclosure provides a precipitation soil erosion and microbial respiration monitoring device, including a precipitation regulation unit, an erosion collection unit, a precipitation monitoring unit, and a microbial respiration monitoring unit; the precipitation monitoring unit is arranged on one side of the precipitation regulation unit, the erosion collection unit is arranged below one side of the precipitation regulation unit, the precipitation regulation unit is configured to adjust precipitation to simulate a erosion of different amounts of precipitation on the soil; the microbial respiration monitoring unit is configured to monitor a microbial respiration in the soil.

In some embodiments, the precipitation regulation unit includes a water collection groove, baffles, and a water storage tank, multiple baffles are provided and arranged in an array above the water collection groove, the water collection port is arranged on one side of the water collection groove, the water collection port is connected to the water storage tank by a connecting hose, a water pump is arranged on the connecting hose, a stand is arranged below one side of the water collection groove, and a retractable adjustment bracket is arranged below the other side.

In some embodiments, the baffle is arranged to be circular, and a buckle is arranged on one side of the baffle; the baffle is connected to the water collection groove through the buckle.

In some embodiments, the erosion collection unit includes an erosion collection box, an erosion soil particle guide plate, filter screens and a mass sensor; the erosion collection box is arranged on one side of the stand and is connected to the stand, the erosion soil particle guide plate is connected to a top of the erosion collection box on one side, and the other side is connected to a bottom of the retractable adjustment bracket, the erosion collection box is equipped with a collection port, multiple collection boxes are vertically equipped in the erosion collection box, multiple filter screens are provided and are respectively arranged above the collection box of each layer, pores of the filter screens decrease from top to bottom, the mass sensor is arranged at a bottom of the collection box of each layer.

In some embodiments, the microbial respiration monitoring unit includes a layered sampler and a microbial respiration monitoring component; the microbial respiration monitoring component is arranged inside the layered sampler, and the microbial respiration monitoring component includes a CO₂ sensor and a temperature and humidity sensor.

In some embodiments, the layered sampler includes a shell, an inner part of the shell is evenly arranged with several partitions from top to bottom to form multiple sampling spaces, each sampling space is equipped with a sampling component, the sampling component includes a telescopic manipulator and a soil drill, the soil drill is connected to a driving end of the telescopic manipulator, a fixed end of the telescopic manipulator is fixedly connected to an inner wall of the shell, multiple automatic opening and closing doors are opened on the shell, a count of the automatic opening and closing door is the same as a count of sampling spaces, and corresponds to the telescopic manipulator, which is configured to control a sampling of the sampling components in the sampling space of each layer.

In some embodiments, a soil sample collection container is arranged in each sampling space, the CO₂ sensor and the temperature and humidity sensor are arranged above the soil sample collection container, and the soil sample collection container is arranged below the sampling component.

In some embodiments, a controller is arranged above the shell to control a sampling device and the automatic opening and closing door.

In some embodiments, the precipitation monitoring unit includes a high-precision rain gauge.

In some embodiments, it also includes a wireless transmission unit, the wireless transmission unit is connected to the precipitation monitoring unit, the erosion collection unit, and the microbial respiration monitoring unit.

Therefore, the disclosure uses the above-mentioned precipitation soil erosion and microbial respiration monitoring device to realize the simultaneous monitoring of soil erosion and microbial respiration, and provides basic data for an in-depth understanding of soil environmental dynamics.

The following is a further detailed description of the technical scheme of the disclosure through drawings and implementation examples.

### BRIEF DESCRIPTION OF THE DRAWINGS

FIG. 1 is a structural diagram of the embodiment of the disclosure;

FIG. 2 is a top view of the precipitation regulation unit of the embodiment of the disclosure;

FIG. 3 is a side view of the precipitation regulation unit of the embodiment of the disclosure;

FIG. 4 is an internal schematic diagram of the sampler of the embodiment of the disclosure;

FIG. 5 is a cross-sectional view of the sampler of the embodiment of the disclosure;

Marks in the FIG.
1, wireless transmission unit; 2, rain gauge; 3, water collection groove; 4, water collection port; 5, water pump; 6, water storage tank; 7, erosion collection box; 71, collection port; 8, sampler; 81, shell; 82, partition; 9. retractable adjustment bracket; 10, baffle; 11, buckle; 12, stand; 13, connecting hose; 14, erosion soil particle guide plate; 15, filter screen; 16, mass sensor; 17, sprinkler irrigation component; 18, controller; 19, sampling component; 20, telescopic manipulator; 21, soil drill; 22, soil sample collection container; 23, CO₂ sensor; 24, temperature and humidity sensor; 25, automatic opening and closing door.

### DETAILED DESCRIPTION

In order to make the purpose, technical scheme, and advantages of the embodiment of the disclosure clearer, the following will describe the technical scheme of the embodiment of the disclosure clearly and completely in combination with the attached figures of the embodiment of the disclosure. Obviously, the described embodiment is part of the embodiment of the disclosure, not all of the embodiment. The components of the embodiment of the disclosure, usually described and illustrated in the accompanying diagram here, can be arranged and designed in a variety of different configurations. In the description of the disclosure, it is necessary to explain that the orientation or positional relationship indicated by the terms ‘up’, ‘down’, ‘inside’, ‘outside’, etc. is based on the orientation or positional relationship shown in the attached diagram, or is the orientation or positional relationship that is habitually placed when the disclosure product is used, only to facilitate the description of the disclosure and simplify the description, rather than to indicate or imply that the device or component referred to must have a specific orientation, be constructed and operated in a specific orientation, and therefore cannot be understood as a limitation to the disclosure.

### Embodiment

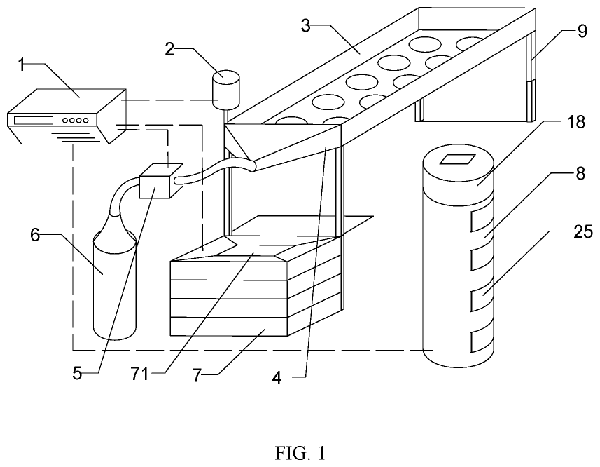

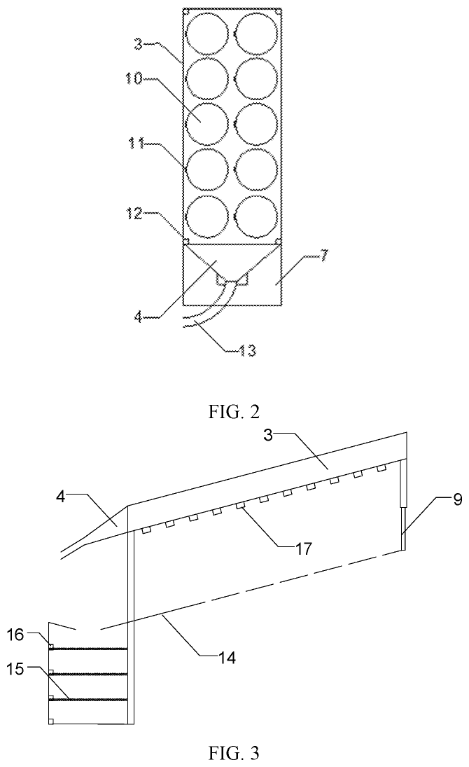

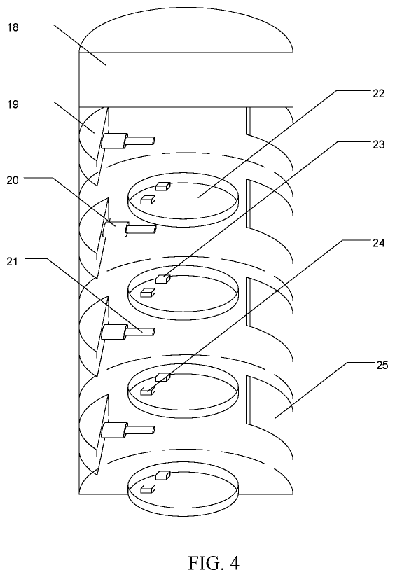

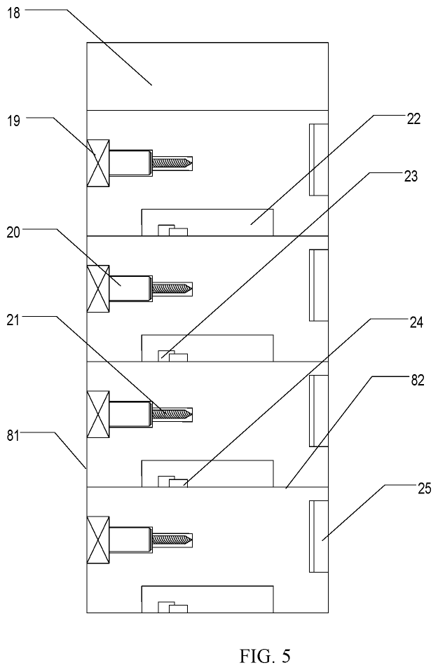

Referring to FIG. 1-FIG. 5, the disclosure provides a precipitation soil erosion and microbial respiration monitoring device, including a precipitation regulation unit, an erosion collection unit, a precipitation monitoring unit, and a microbial respiration monitoring unit; the precipitation monitoring unit is arranged on the side of the precipitation regulation unit, and the erosion collection unit is arranged below the side of the precipitation regulation unit; the precipitation regulation unit is configured to adjust the precipitation to simulate the erosion of different precipitation on the soil, and the microbial respiration monitoring unit is configured to monitor the microbial respiration in the soil.

The precipitation regulation unit includes a water collection groove 3, a baffle 10, and a water storage tank 6; multiple baffles 10 are provided and arranged in an array above the water collection groove 3; the water collection port 4 is arranged on one side of the water collection groove 3, the water collection port 4 is connected to the water storage tank 6 by a connecting hose 13, a water pump 5 is arranged on the connecting hose 13, a stand 12 is arranged below one side of the water collection groove 3, and a retractable adjustment bracket 9 is arranged below the other side.

The baffle 10 is arranged to be circular, and the side of the baffle 10 is provided with a buckle 11, which is connected to the water collection groove 3 through the buckle 11. When regulating precipitation, the amount of water flowing into the soil is controlled by adjusting the opening and closing of different numbers of baffles 10 to simulate the reduced precipitation. For example, for the experimental point of reducing precipitation, the baffle 10 opening is arranged to 75% to simulate a 25% reduction in precipitation conditions. A water collection port 4 is arranged on one side of the water collection groove 3, which collects the precipitation blocked by the baffles 10, and the water is transported to the water storage tank 6 through the pump 5 and the connecting hose 13 for storage. If you need to increase precipitation, you can use artificial precipitation enhancement devices, such as the use of sprinkler irrigation component 17, to simulate increased precipitation conditions. The sprinkler irrigation component 17 is arranged below the water collection groove 3.

The erosion collection unit includes an erosion collection box 7, an erosion soil particle guide plate 14, filter screens 15 and a mass sensor 16; the erosion collection box 7 is arranged on one side of the stand 12 and is connected to the stand 12, the erosion soil particle guide plate 14 is connected to a top of the erosion collection box 7 on one side, and the other side is connected to the bottom of the retractable adjustment bracket 9, the erosion collection box 7 is equipped with a collection port 71, multiple collection boxes are vertically equipped in the erosion collection box 7, multiple filter screens 15 are provided and are respectively arranged above the collection box of each layer, pores of the filter screens 15 decrease from top to bottom, the mass sensor 16 is arranged at the bottom of the collection box of each layer to record the quality of soil erosion materials with different particle sizes.

The precipitation monitoring unit includes a high-precision rain gauge 2, the high-precision rain gauge 2 is arranged on one side of the water collection groove 3 for real-time monitoring of precipitation.

The microbial respiration monitoring unit includes a layered sampler 8 and a microbial respiration monitoring component, the microbial respiration monitoring component is arranged inside the layered sampler 8, the microbial respiration monitoring component includes a CO₂ sensor 23 and a temperature and humidity sensor 24.

The layered sampler 8 includes a shell 81, and multiple partitions 82 are uniformly arranged inside the shell 81 from top to bottom to form multiple sampling spaces, a sampling component 19 is arranged in each sampling space, the sampling component 19 includes a telescopic manipulator 20 and a soil drill 21, the soil drill 21 is connected to the drive end of the telescopic manipulator 20, and the fixed end of the telescopic manipulator 20 is fixedly connected to the inner wall of the shell 81. The shell 81 is equipped with multiple automatic opening and closing doors 25. The count of the automatic opening and closing door 25 is the same as the count of sampling spaces and corresponds to the telescopic manipulator 20, which is configured to control the sampling of the sampling component 19 in each sampling space.

Each sampling space is equipped with a soil sample collection container 22, a CO₂ sensor 23, and a temperature and humidity sensor 24 are set above the soil sample collection container 22 for real-time monitoring of CO₂ concentration changes, soil temperature, and humidity generated by microbial activities.

A controller 18 is arranged above the shell 81, which is configured to control the sampling device and the automatic opening and closing door 25.

It also includes a wireless transmission unit 1 and a GPS module; the wireless transmission unit 1 is connected to the precipitation monitoring unit, erosion collection unit, and microbial respiration monitoring unit, which is configured to transmit monitoring data of the rain gauge 2, the CO₂ sensor 23, the temperature and humidity sensor 24, and the mass sensor 16.

Considering various complex and changeable climatic conditions and geographical environments, the device is made of low-temperature-resistant, ultraviolet-resistant, and corrosion-resistant materials to adapt to a variety of environments.

Working principle: The sampler 8 is buried in the soil, and the precipitation regulation unit is installed on the slope surface, the height of the precipitation regulation unit is adjusted by the retractable adjustment bracket, and the precipitation regulation unit is controlled to be parallel to the slope surface, where the erosion collection unit is set below the slope.

After the precipitation causes soil erosion, the soil erosion particles move downward from the slope surface. The soil erosion particles are transmitted to the erosion collection box 7 through the erosion soil particle guide plate 14. The erosion collection box 7 collected soil erosion particles through the collection port 71. The soil erosion particles of different particle sizes were collected by filter screen 15 with different pore sizes, and the quality of soil erosion materials of various particle sizes was recorded by mass sensor 16.

In the process of soil collection, first of all, the automatic opening and closing door 25 is opened, and the telescopic manipulator 20 is extended to the outside of the sampler 8. The soil sample is collected by the soil drill 21, and the collected soil sample is stored in the soil sample collection container 22. After the collection is completed, the automatic opening and closing door 25 is closed to avoid the gas exchange inside and outside the sampler 8, and a closed environment is formed in each sampling layer to capture and analyze the CO₂ produced by soil microorganisms, so as to more accurately evaluate microbial respiration.

Meanwhile, the precipitation is monitored in real time by the high-precision rain gauge 2.

Finally, the precipitation data, CO₂ monitoring data, temperature and humidity monitoring data, and quality data are transmitted through wireless transmission unit 1. By analyzing the collected data, the amount of soil erosion caused by precipitation, the changes in soil microbial activity, and the interaction between them can be obtained.

Therefore, the disclosure uses the above-mentioned precipitation soil erosion and microbial respiration monitoring device to realize the simultaneous monitoring of soil erosion and microbial respiration, and provides basic data for an in-depth understanding of soil environmental dynamics.

Finally, it should be explained that the above embodiments are only used to explain the technical scheme of the disclosure rather than restrict it. Although the disclosure is described in detail with reference to the better embodiment, the ordinary technical personnel in this field should understand that they can still modify or replace the technical scheme of the disclosure, and these modifications or equivalent substitutions cannot make the modified technical scheme out of the spirit and scope of the technical scheme of the disclosure.

What is claimed is:

1. A precipitation soil erosion and microbial respiration monitoring device, comprising a precipitation regulation unit, an erosion collection unit, a precipitation monitoring unit, and a microbial respiration monitoring unit; the precipitation monitoring unit is arranged on one side of the precipitation regulation unit, the erosion collection unit is arranged below one side of the precipitation regulation unit, the precipitation regulation unit is configured to adjust precipitation to simulate a erosion of different amounts of precipitation on the soil; the microbial respiration monitoring unit is configured to monitor a microbial respiration in the soil.

2. The precipitation soil erosion and microbial respiration monitoring device according to claim 1, wherein the precipitation regulation unit comprises a water collection groove, baffles, and a water storage tank, multiple baffles are provided and arranged in an array above the water collection groove, the water collection port is arranged on one side of the water collection groove, the water collection port is connected to the water storage tank by a connecting hose, a water pump is arranged on the connecting hose, a stand is arranged below one side of the water collection groove, and a retractable adjustment bracket is arranged below the other side.

3. The precipitation soil erosion and microbial respiration monitoring device according to claim 2, wherein the baffle is arranged to be circular, and a buckle is arranged on one side of the baffle, the baffle is connected to the water collection groove through the buckle.

4. The precipitation soil erosion and microbial respiration monitoring device according to claim 2, wherein the erosion collection unit comprises an erosion collection box, an erosion soil particle guide plate, filter screens and a mass sensor; the erosion collection box is arranged on one side of the stand and is connected to the stand, the erosion soil particle guide plate is connected to a top of the erosion collection box on one side, and the other side is connected to a bottom of the retractable adjustment bracket, the erosion collection box is equipped with a collection port, multiple collection boxes are vertically equipped in the erosion collection box, multiple filter screens are provided and are respectively arranged above the collection box of each layer, pores of the filter screens decrease from top to bottom, the mass sensor is arranged at a bottom of the collection box of each layer.

5. The precipitation soil erosion and microbial respiration monitoring device according to claim 1, wherein the microbial respiration monitoring unit comprises a layered sampler and a microbial respiration monitoring component, the microbial respiration monitoring component is arranged inside the layered sampler, the microbial respiration monitoring component comprises a CO₂ sensor and a temperature and humidity sensor.

6. The precipitation soil erosion and microbial respiration monitoring device according to claim 2, wherein the layered sampler comprises a shell, an inner part of the shell is evenly arranged with several partitions from top to bottom to form multiple sampling spaces, each sampling space is equipped with a sampling component, the sampling component comprises a telescopic manipulator and a soil drill, the soil drill is connected to a driving end of the telescopic manipulator, a fixed end of the telescopic manipulator is fixedly connected to an inner wall of the shell, multiple automatic opening and closing doors are opened on the shell, a count of the automatic opening and closing door is the same as a count of sampling spaces, and corresponds to the telescopic manipulator, which is configured to control a sampling of the sampling components in the sampling space of each layer.

7. The precipitation soil erosion and microbial respiration monitoring device according to claim 6, wherein a soil sample collection container is arranged in each sampling space, the CO₂ sensor and the temperature and humidity sensor are arranged above the soil sample collection container, and the soil sample collection container is arranged below the sampling component.

8. The precipitation soil erosion and microbial respiration monitoring device according to claim 7, wherein a controller is arranged above the shell to control a sampling device and the automatic opening and closing door.

9. The precipitation soil erosion and microbial respiration monitoring device according to claim 1, wherein the precipitation monitoring unit comprises a high-precision rain gauge.

10. The precipitation soil erosion and microbial respiration monitoring device according to claim 1, wherein it also comprises a wireless transmission unit, the wireless transmission unit is connected to the precipitation monitoring unit, the erosion collection unit, and the microbial respiration monitoring unit.
